(19) United States
(12) Patent Application Publication
(10) Pub. No.: US 2015/0219617 A1
(43) Pub. Date: Aug. 6, 2015

(54) Device For The Optical Inspection Of A Moving Textile Material

(71) Applicant: Uster Technologies AG, Uster (CH)

(72) Inventors: Rafael Storz, Kreuzlingen (CH); Peter Pirani, Grut (CH)

(73) Assignee: USTER TECHNOLOGIES AG, Uster (CH)

(21) Appl. No.: 14/688,411

(22) Filed: Apr. 16, 2015

Related U.S. Application Data
(63) Continuation of application No. PCT/CH2013/000209, filed on Dec. 2, 2013.

(30) Foreign Application Priority Data
Dec. 10, 2012 (CH) 2739/12

(51) Int. Cl. G01N 33/36; G01N 21/88; G01N 21/84

(52) U.S. Cl. CPC G01N 33/365; G01N 21/84; G01N 21/8851; G01N 2201/062; G01N 2201/0612; G01N 2201/08; G01N 2201/10

(57) ABSTRACT

Using a substrate with at least one optical waveguide structure integrated thereon for the optical inspection of a moving textile material. A scanning region is provided on the substrate for optically scanning the textile material. The optical waveguide structure opens at least partly into the scanning region. The device arranged in this manner requires little space, can be used in a versatile manner and can be changed or maintained with very little effort.

This application is a continuation application of prior pending PCT patent application PCT/CH2013/000209 filed 2013 Dec. 2, and claims priority on Swiss patent application 2739/12 filed 2012 Dec. 10.

### BACKGROUND

The present invention lies in the field of textile material testing. It relates to a device and a method for the optical inspection of a moving textile material, according to the preambles of the independent claims. The invention can be used for example in yarn testing devices in the textile laboratory or in yarn clearers on spinning or winding machines.

A large number of different devices are known for testing textile materials. Different sensor principles are used in the textile testing devices. The use of a specific sensor principle depends among other things on the property that needs to be detected optimally. Frequently used sensor principles, especially in yarn testing, are the following:

The capacitive sensor principle; cf. U.S. Pat. No. 6,346,819 B1. The textile material is guided through an air gap of a measuring capacitor. The measuring capacitor substantially measures the mass of the textile material contained in said capacitor. The capacitive sensor principle offers high measuring precision and a sensitivity which is stable over a long period of time. Its disadvantages are an undesirable sensitivity to changes in humidity and non-usability with electrically conductive textile materials.

The optical sensor principle; cf. WO-2004/044579 A1. The textile material is illuminated by a light source and light interacting with the textile material is detected by light detectors. The detected light is a measure for the diameter of the textile material and/or its optical properties such as reflectivity or color. The optical sensor principle is less sensitive to changes in diameter and less stable in the long term than the capacitive one. It can nevertheless be advantageous, especially for such applications for which the capacitive sensor principle is unsuitable, e.g. in environments with strong fluctuations in humidity or for electrically conductive textile materials. Foreign substances which have a reflectivity which deviates strongly from the textile material can be detected in a simple manner by the optical sensor principle.

It has already been proposed to scan yarn with different sensor principles and to combine the sensor signals in the evaluation with each other. CN-2'896'282 Y thus mentions the combination of a capacitive and a photoelectric sensor for detecting the mass density and the diameter of the same yarn. WO-01/92875 A1 teaches the arrangement of two sensors in succession along the yarn path. A first one of the sensors measures the optical reflection on the yarn. A second one of the sensors capacitively or optically measures the mass or the diameter of the yarn. The output signals of the two sensors are evaluated according to specific evaluation criteria. At least two types of foreign substances can be distinguished from each other on the basis of the evaluation.

WO-93/13407 A1 provides an example for an optical yarn clearer measuring head for the detection of foreign fibers. The yarn that is moved through a measuring slit is illuminated by a light source with modulated light. A first sensor receives light reflected from the yarn and at the same time a second sensor receives light transmitted from the yarn. Conclusions on the presence of a foreign fiber in the yarn are drawn from the electrical signals that are output by the two sensors. Three-dimensional light feeders are provided for guiding light between the light source and the sensors and the measuring slit, which light feeders are arranged for example as hollow cavities that are mirror-coated on the inside.

U.S. Pat. No. 5,768,938 A reduces the need for space of the measuring head in comparison with WO-93/13407 A1, in that the light feeders are arranged in a plane which stands perpendicularly to the yarn. The light feeders are arranged as a three-dimensional body which transmits light and which is inserted into a three-dimensional base body. The base body also comprises a receiving opening for a light source. Even this apparatus still requires a relatively large amount of space. If a change in the optical scanning part is necessary, the entire measuring head would have to be newly designed and the respective production tools would have to be newly constructed, which is exceedingly laborious.

DE-38'30'665 A1 discloses an optoelectronic apparatus for thread monitoring. All active optoelectronic components such as light-emitting diodes and photo transistors are attached to a central unit. The central unit is connected by means of optical waveguides to several yarn stop motions, which are respectively situated at a thread running point. A yarn stop motion only consists of a circuit board with a thread guide eyelet. The ends of two optical waveguides connected to the central unit are inserted into a respective opening in the circuit board in such a way that they are situated opposite of each other.

An optoelectronic yarn sensor is known from DE-10'2007'040'224 A1. The components of the yarn sensor are arranged on a circuit board. An emitter diode emits light which is directed by a lens to the yarn. The light transmitted by the yarn is detected by a receiver diode. A portion of the light emitted by the emitter diode is split off by the lens and is supplied by an optical waveguide integrated in the lens to a monitor diode. The current of the emitter diode is controlled depending on the signal of the monitor diode, so that a constant emitted luminous intensity is obtained.

The circuit boards with integrated optical waveguide structures and integrated electrical conductor structures are generally known, e.g. from US-2010/0209854 A1.

### SUMMARY

It is an object of the present invention to provide a device for the optical scanning of a moving textile material which requires less space than the devices known from the state of the art. Furthermore, the device shall be usable in a versatile manner and can be changed or maintained with little effort.

These and other objects are achieved by the device in accordance with the invention as defined in the first claim. Advantageous embodiments are provided in the dependent claims.

In accordance with the invention, a substrate is used having at least one optical waveguide structure integrated thereon for the optical inspection of a moving, preferably elongated, textile material. An electrical conductor structure for electrically testing the moved textile material can additionally be integrated on the substrate.

A waveguide structure which is accommodated monolithically in or on the substrate shall be understood in this specification as an optical waveguide structure integrated on a substrate. The waveguide structure was originally produced on the substrate, e.g. by technologies such as photolithography and/or doping, which is in contrast to separate, discrete waveguides which are put subsequently on a substrate. The integrated optical waveguide structure is inseparably connected to the substrate. It contains a plurality of transparent dielectric layers with different refractive indexes. A core layer with a higher refractive index is embedded between an upper and a bottom layer with lower refractive indexes, so that light waves can be guided in the core layer. The waveguide structure can preferably contain micro strip waveguides which guide light in one direction and/or flat thin-layer waveguides in which light can propagate in two directions. In addition to the waveguides, it can contain further passive and/or active integrated optical components such as lenses, beam splitters, reflectors, filters, amplifiers, light sources and/or light receivers.

The device in accordance with the invention for the optical inspection of a moving, preferably elongated textile material also contains a substrate on which a scanning region for the optical scanning of the textile material is provided, and an optical waveguide structure which is integrated on the substrate and which opens at least partly into the scanning region.

In a preferred embodiment, at least two optical waveguides of the optical waveguide structure open into the scanning region. At least two waveguides can be arranged for guiding light towards the scanning region and at least two waveguides for guiding light away from the scanning region. The orifices of the waveguides that guide towards and away from the scanning region are preferably arranged in an alternating fashion adjacent to each other.

It is advantageous for avoiding light losses if at least one orifice of a waveguide to the scanning region is provided with a focusing lens.

The optical waveguide structure can comprise at least one junction with at least two branches. At least two of the branches can face the scanning region or face away from the scanning region.

In a further preferred embodiment, the substrate comprises at least one optical interface outside of the scanning region for connecting the at least one optical waveguide structure to one respective optical connecting part. The at least one interface preferably comprises mechanical positioning means for positioning the optical connecting part with respect to the substrate. An incoupling interface can be provided for incoupling light into at least one optical waveguide structure and an outcoupling interface which differs from the incoupling interface for outcoupling light from at least one optical waveguide structure. The at least one optical interface is preferably arranged for connecting at least two optical waveguides to an optical connecting part. It is preferably attached to an edge of the substrate.

The invention also relates to a combination of a device in accordance with the invention, which comprises the aforementioned at least one optical interface, with an optical connecting part. An incoupling connecting part which is associated with the incoupling interface can contain a row of at least two light sources, preferably a light-emitting diode array, and an outcoupling connecting part which is associated with the outcoupling interface can contain a row of at least two light receivers, preferably a CCD array.

According to a further preferred embodiment, an electrical conductor structure is additionally integrated on the substrate, which structure opens at least partly into the scanning region. The textile material can thus selectively be examined in an optical, electrical or both optical and also electrical manner. Advantageously, at least two electrical conductor paths of the electrical conductor structure open into the scanning region. The orifices of the optical waveguides and the orifices of the electrical conductor paths can be arranged in an alternating fashion adjacent to each other. At least one orifice of an electrical conductor path is preferably provided with an electrode in the scanning region. The substrate can comprise at least one electrical interface for connecting the at least one electrical conductor structure to one respective electrical connecting part outside of the scanning region. The at least one electrical interface preferably comprises mechanical positioning means for positioning the electrical connecting part with respect to the substrate. It is attached to an edge of the substrate for example.

In the case of devices having a substrate with an optical waveguide structure and an electrical conductor structure, the substrate can comprise at least one optical-electric interface for connecting the at least one optical waveguide structure and the at least one electrical conductor structure to a respective optical-electrical connecting part outside of the scanning region. The at least one optical-electrical interface preferably comprises mechanical positioning means for positioning the optical-electrical connecting part with respect to the substrate.

The integration of the optical waveguide structure on the substrate leads to space saving with respect to known optical devices for testing textile materials. Furthermore, the substrate can be exchanged easily in the device in accordance with the invention in order to maintain or change the device. A change in the device can occur by the replacement of a specific substrate by another substrate in that the scanning region for example is arranged differently. A first substrate can be provided for example by means of which the textile material is examined from only one side, and a second substrate by means of which the textile material is examined from several directions along its circumference.

In the present specification, the terms such as “light” or “illuminating” are not only used for visible light, but also for electromagnetic radiation from the adjacent spectral ranges of ultraviolet (UV) and infrared (IR).

### DRAWINGS

The invention is explained below in closer detail by reference to the schematic drawings, wherein:

FIGS. 1 to 4 schematically show four different embodiments of a device in accordance with the invention in top views;

FIG. 5 schematically shows one end of an optical waveguide, a textile material to be tested and light beams extending in between in a top view;

FIGS. 6 to 9 schematically show four further embodiments of a device in accordance with the invention in top views;

FIGS. 10 and 11 schematically show two further embodiments of a device in accordance with the invention in perspective views;

FIG. 12 schematically shows an eleventh embodiment of a device in accordance with the invention in a cross-sectional view.

### DESCRIPTION

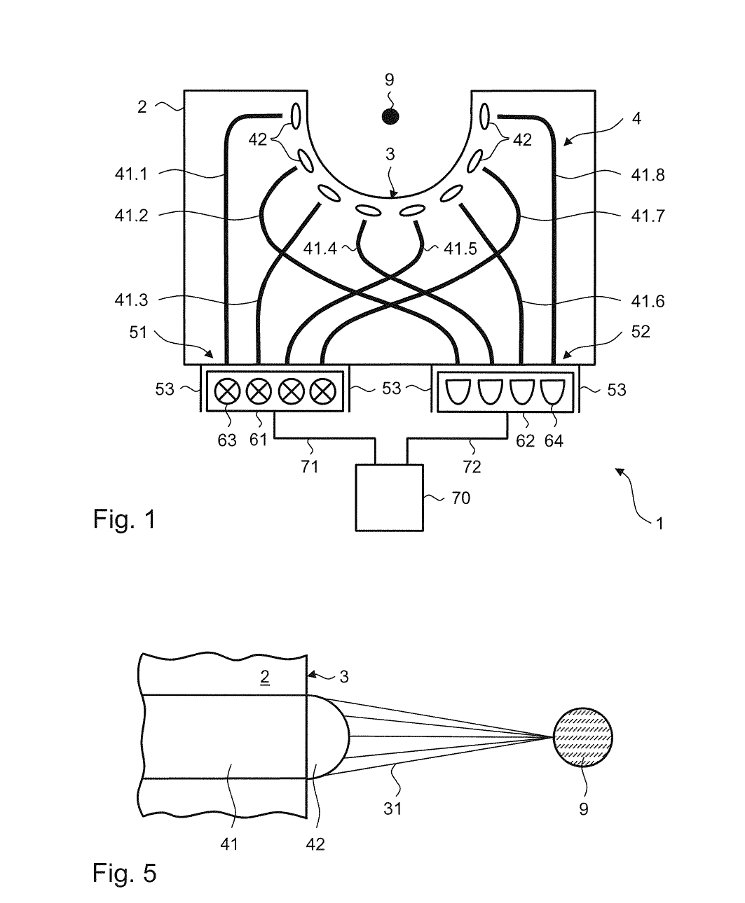

FIG. 1 shows a first embodiment of a device 1 in accordance with the invention. The device 1 is used for testing a preferably elongated textile material 9, e.g. a yarn, which is moved through the device 1 or past the device 1. In the present case, the direction of movement of the textile material 9 extends perpendicularly to the plane of the drawing, in the direction of a longitudinal axis of the textile material 9. The device 1 contains a substrate 2, on which a scanning region 3 for the optical scanning of the textile material 9 is provided. The substrate 2 can consist of a known material such as glass, a synthetic material, a semiconductor material, or a glass fiber mat impregnated with epoxy resin. It is preferably flat and rigid, i.e. it practically does not deform.

The scanning region 3 is arranged in the present embodiment as a substantially semicircular recess at an edge of the substrate 2. The textile material 9 is guided through the device 1 in such a way that its longitudinal axis is situated as close as possible to the center point of the semicircle. The plane of the substrate 2 lies perpendicularly to the longitudinal axis of the textile material 9.

An optical waveguide structure 4 is integrated on the substrate 2 for guiding light towards the scanning region 3 and/or away from the scanning region 3. In the embodiment of FIG. 1, the waveguide structure 4 contains eight optical microstrip waveguides 41.1 to 41.8, which respectively connect the scanning region 3 to an optical interface 51, 52. The waveguide structure 4 can be made of a polymer for example which is sufficiently transparent for the used light wavelength. It is preferably applied to the substrate 2 by a photolithographic process. The transverse dimensions (width and height) of a single waveguide 41.1 to 41.8 can be between 5 μm and 500 μm, preferably approximately 50 μm. The waveguide structure 4 can be situated on an outermost layer of the substrate 2, or form an inner layer which is covered by at least one layer situated on top of said inner layer. In the latter case, the layer situated on top can protect the waveguide structure 4 from mechanical damage, soiling and undesirable optical influences. The waveguides 41.1 to 41.8 can be arranged as single-mode or multimode waveguides. The waveguide structure 4 of FIG. 1 comprises several crossings of waveguides 41.1 to 41.8. Notice shall be taken that crosstalk from the one to the other waveguide is prevented. The person skilled in the art of integrated optics is capable of designing the waveguide structure 4 in such a way that this condition is fulfilled correctly. This can especially be the case when the respective crossing angle is close to 90° or is at least not too acute. In addition to the waveguides 41.1 to 41.8 per se, the integrated optical waveguide structure may contain further integrated optical components such as lenses, beam splitters, reflectors, filters, amplifiers, light sources and/or light receivers.

Light-collecting elements 42 such as focusing lenses are preferably attached in the scanning region 3 to the orifices of the waveguides 41.1 to 41.8 in order to ensure the highest possible light yield. It is known that light exiting from the end of a thin waveguide is emitted in a large opening angle. In temporal reversal, light from the same large opening angle is therefore incoupled into the waveguide. Since the textile material 9 to be tested mostly has a small diameter of less than 1 mm, it would be struck without countermeasures by merely a small portion of the available light, and from said light only a small part would be incoupled into a waveguide again. The focusing lenses 42 are used to avoid such losses of light. Their function and their configuration are explained below in closer detail by reference to FIG. 5.

Four of the eight waveguides 41.1 to 41.8, which are designated below as “illumination waveguides” 41.1, 41.3, 41.5, 41.7, are used for illuminating the textile material 9. For this purpose, they receive light from an emitter module 61 and guide it to the scanning region 3, where it exits from the illumination waveguides 41.1, 41.3, 41.5, 41.7 and impinges on the textile material 9 at least in part. The emitter module 61 can be attached to the end of a first electrical conductor 71. The light transfer from the emitter module 61 to the illumination waveguides 41.1, 41.3, 41.5, 41.7 occurs on an incoupling interface 51 which is attached to an edge of the substrate 2. The incoupling interface 51 can be arranged as a plug-in connection for example. The emitter module 61 contains light sources 63 which are arranged adjacently in a row for example and of which each is assigned to one of the four illumination waveguides 41.1, 41.3, 41.5, 41.7. The light sources 63 can be arranged as diode lasers or light-emitting diodes. The incoupling of the light from the light sources 63 into the illumination waveguides 41.1, 41.3, 41.5, 41.7 can occur by direct illumination of the ends of the illumination waveguides or by means of optical elements such as mirrors and/or focusing lenses 42. In the latter case, similar lenses can be used as in the orifice to the scanning region 3 (see FIG. 5). It is important for ensuring effective incoupling of the light into the illumination waveguides 41.1, 41.3, 41.5, 41.7 at the incoupling interface 51 that the light sources 63 are positioned as precisely and as stable as possible with respect to the ends of the illumination waveguides. For this purpose, mechanical positioning means 53 for positioning the emitter module 61 with respect to the substrate 2 are preferably attached to the incoupling interface 51. The positioning means 53 can be arranged as suitable guides for example, which ensure precise positioning within the plug-in connection. They are merely schematically indicated in FIG. 1, like the other elements.

The other four of the eight waveguides 41.1 to 41.8 are used for detecting the light reflected from the textile material 9 or transmitted past said material, and are therefore designated below as “detection waveguides” 41.2, 41.4, 41.6, 41.8. They guide the light coming from the scanning region 3 to a receiver module 62, which can be attached to the end of a second electric conductor 72. The light transfer from the detection waveguides 41.2, 41.4, 41.6, 41.8 to the receiver module 62 occurs at an outcoupling interface 52 which is attached to an edge of the substrate 2. The outcoupling interface 52 can also be arranged as a plug-in connection with respective positioning means 53. The receiver module 62 contains light receivers 64 which are arranged in a row adjacent to each other for example and each of which is assigned to one of the four detection waveguides 41.2, 41.4, 41.6, 41.8. The row of light receivers can be arranged as a CCD array for example. It is also possible to combine several receiver elements situated adjacent to each other, which then form an “assembled light receiver” and are assigned to a detection waveguide 41.2, 41.4, 41.6, 41.8. Concerning light outcoupling and the positioning and arrangement of the outcoupling interface 52, the same applies as already discussed analogously with respect to the incoupling interface 51.

The emitter module 61 and the receiver module 62 are connected via the first electrical conductor 71 and the second electrical conductor 72 to an electronic unit 70. It triggers the emitter module 61 on the one hand, and on the other hand the electronic unit 70 receives signals from the receiver module 62, evaluates them itself or conducts them, after optional preprocessing, to an evaluation unit (not shown).

It is advantageous to precisely define the incoupling interface 51 and the outcoupling interface 52 in an optical and mechanical manner and to thus quasi standardize them. As a result, the emitter module 61 and the receiver module 62 with their relatively expensive optoelectronic components can be used without any changes for various substrates 2. On the other hand, the relatively inexpensive substrates 2 with their integrated optical waveguide structures 4 can be exchanged as required. There may be a need for exchanging a substrate 2 for example if a different waveguide structure 4 (especially in the scanning region 3) is needed or if a substrate 2 is damaged by wear and tear or is defective for other reasons.

The device 1 in accordance with the invention is preferably housed in a housing as known for example from U.S. Pat. No. 5,768,938 A. Such a housing was not included in the enclosed drawings for reasons of clarity of the illustration.

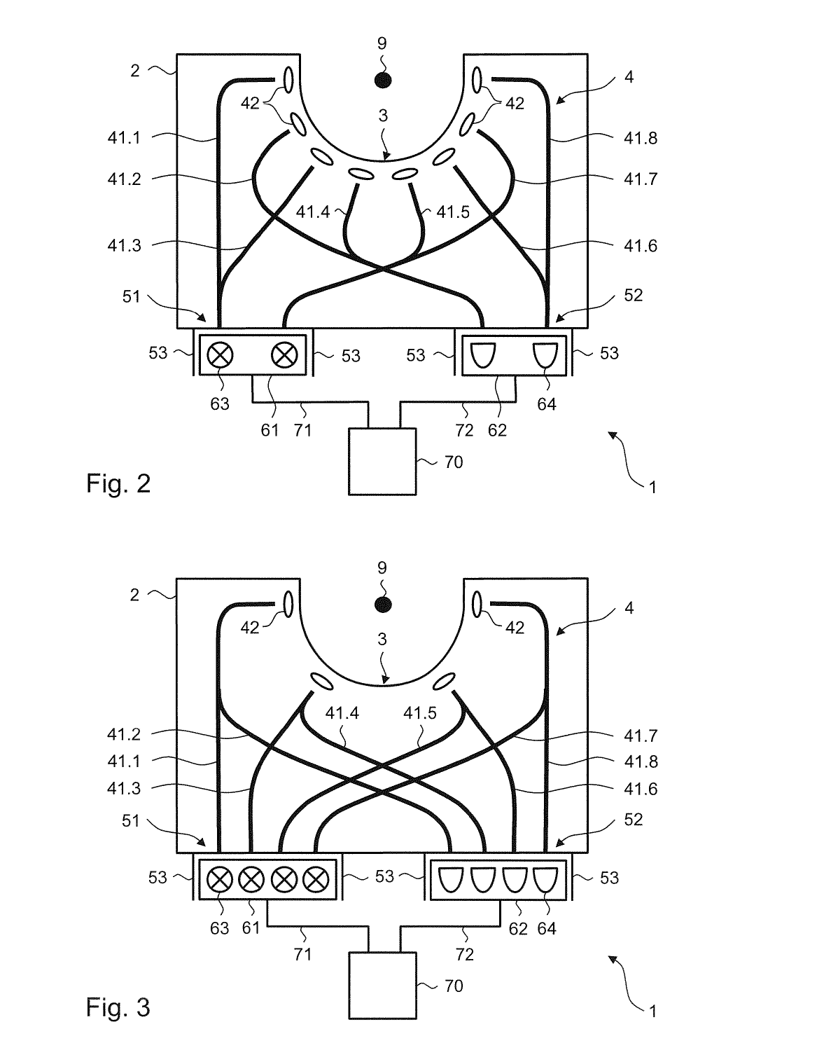

FIG. 2 shows a second embodiment of the device 1 in accordance with the invention. It is simplified with respect to the first embodiment of FIG. 1 in the respect that the emitter module 61 comprises only two light sources 63 and the receiver module 62 only two light receivers 64. The number of the ends of the waveguides opening into the scanning region 3 is the same as in the first embodiment. This is possible by using junctions in the waveguide structure 4. Each of the four waveguides facing the emitter module 61 and the receiver module 62 comprises a Y-junction whose two branches respectively face the scanning region 3, so that twice as many ends of waveguides are situated in the scanning region 3 than in the modules 61, 62. The device 1 could make do with even only one light source and/or one light receiver by using further junctions. The mentioned simplification of the modules 61, 62 is achieved by a lower resolution. The signals of two detection waveguides 41.2, 41.4; 41.6, 41.8 each are combined into a single signal and can only be detected jointly. This need not be disadvantageous however. If it is intended to find foreign fibers in the textile material 9 for example, only the total reflectivity of the textile material 9 along its circumference or a portion thereof is of interest. The second embodiment is similarly suitable like the first one for such an application.

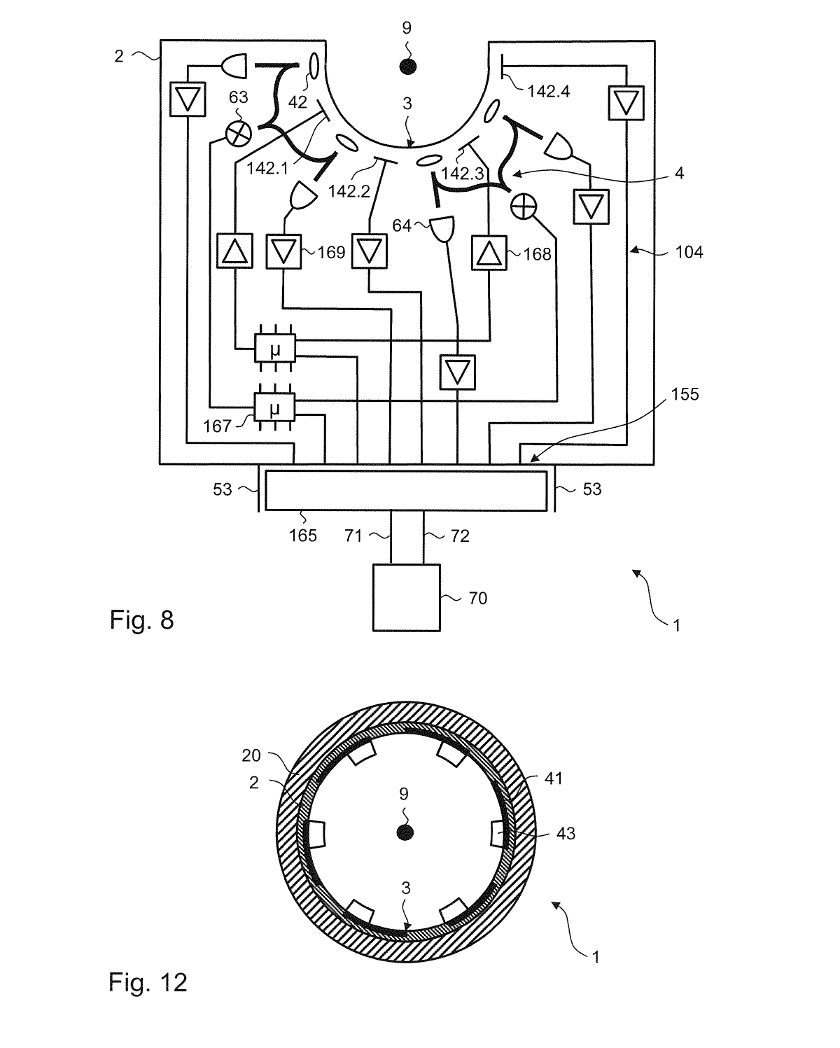

The junctions can be arranged as generally known junction components. Similar to the example of FIG. 2, 1×2 junctions or junctions of a higher order can be concerned. In addition to the junctions, the optical waveguide structure 4 can contain further integrated optical components. They can be passive and/or active. Examples for such integrated optical components are lenses, beam splitters, reflectors, filters, amplifiers, light sources and light receivers. Furthermore, optical elements such as light sources and/or light receivers can be applied as separate discrete components to the substrate 2 (see FIG. 8 in this respect).

A third embodiment of the device 1 in accordance with the invention, which also contains a Y-junction, is shown in FIG. 3. In this case, the simplification over the first embodiment of FIG. 1 consists of halving the number of the ends of the waveguides facing the scanning region 3 without reducing resolution. This is achieved in that each end of a waveguide facing the scanning region 3 is used both for illumination as well as detection. Such a multiple use of a waveguide section is possible because the illumination light and the detection light in the same waveguide section do not influence each other. The junctions are arranged in such a way that a large portion of the light emitted by the associated light source 63 is emitted to the scanning region 3, and a large portion of the light received by the scanning region 3 is supplied to the associated light receiver 64. The person skilled in the art of integrated optics is able to produce such junctions with knowledge of the invention.

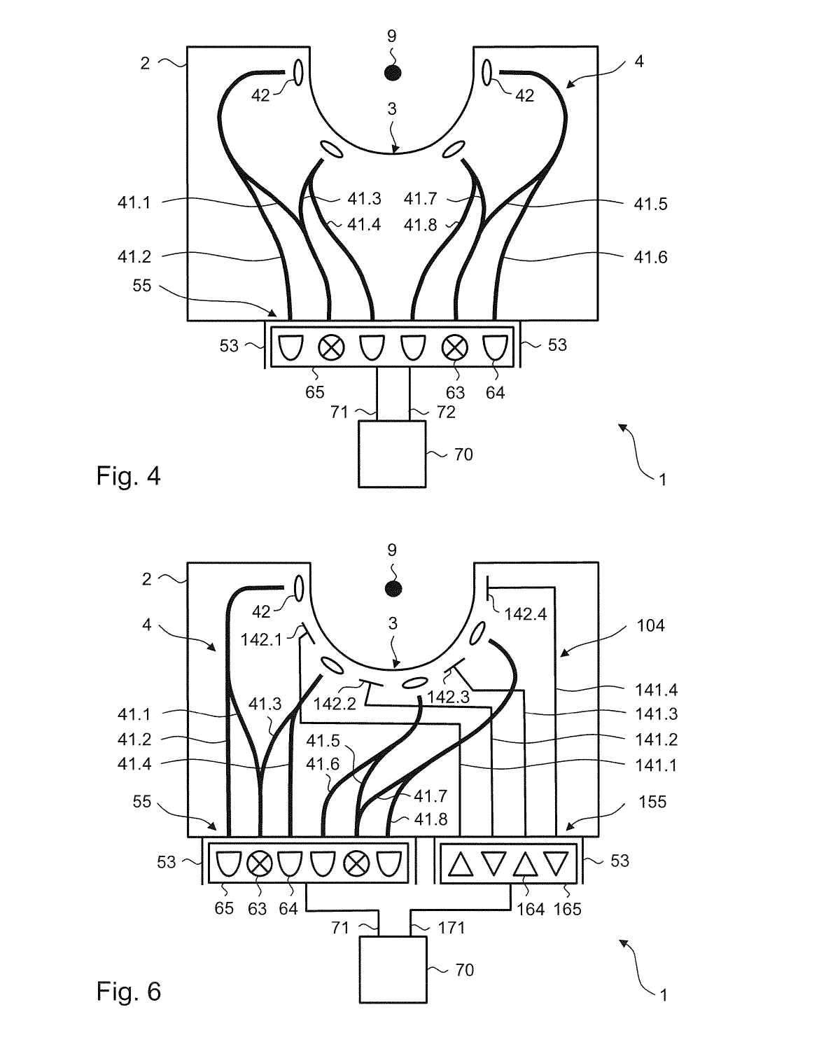

FIG. 4 shows a fourth embodiment of the device 1 in accordance with the invention. In this case, Y-junctions are used to halve the number of required light sources 63 on the one hand and the number of the ends of waveguides facing the scanning region 3 on the other hand with respect to the first embodiment of FIG. 1 without losing resolution. The lack of waveguide crossings is a further advantage of this embodiment. Only one single optical interface 55 is present, which is used both as an incoupling interface and also as an outcoupling interface. Both light sources 63 and also light receivers 64 are situated in the respective transmitter and receiver module 65.

FIG. 5 schematically shows in a highly enlarged view an end of a waveguide 41 integrated on a substrate, which end faces the scanning region 3. It is irrelevant whether an illumination waveguide or a detection waveguide is concerned, because the two cases converge into each other by time reversal. The waveguide end is provided with a focusing lens 42. Focusing lens 42 can be made of the waveguide end itself, be directly glued thereon or spaced therefrom. It is configured and arranged in such a way that it allows the highest possible amount of light 31 exiting from the waveguide 42 to impinge on the textile material 9 or allows the largest possible amount of light 31 coming from the textile material 9 to enter the waveguide 41. The single focusing lens 42 schematically shown in FIG. 5 can be a lens system in practice. The person skilled in the art of technical optics is able with knowledge of the invention to determine and use an arrangement suitable for the purpose as described above.

FIG. 6 shows a fifth embodiment of the device 1 in accordance with the invention. In this case, both an optical waveguide structure 4 and also an electrical conductor structure 104 are integrated on the same substrate 2, wherein both structures 4, 104 open at least partly into the scanning region 3. As a result, this embodiment thus offers the possibility to test the textile material 9 in an optionally optical, electrical, or both optical and also electrical manner.

The optical waveguide structure 4 and the optical interface 51 in the embodiment of FIG. 6 correspond to those of the embodiment of FIG. 4. Only one connecting line between the optical transmitter and receiver module 65 and the electronic unit 70 is shown for reasons of simplicity, wherein several connecting lines may be present however.

The electrical conductor structure 104 is arranged in a very simple way in FIG. 6. It contains four electrically mutually insulated electrical conductor paths 141.1 to 141.4, which respectively lead from the scanning region 3 to an electrical interface 155. Electrical circuits can generally be integrated on the substrate 2, as known from the field of electronics. Such circuits can contain passive and/or active electrical components in addition to electrical conductor paths. Examples for such electrical components are resistors, capacitors, coils, transistors, filters and amplifiers. Complex components such as microprocessors can be situated on the substrate 2, wherein they are preferably put as integrated circuits in a separate housing on the substrate 2 (see FIG. 8 in this respect).

The orifices of the electrical conductor paths 141.1 to 141.4 in the scanning region 3 are provided with electrodes 142. The electrodes 142 are used to produce and/or detect an electrical field, preferably an alternating electrical field, in the scanning region 3. The textile material 9 interacts with the electrical field and influences it. The electrical testing of the textile material 9 is based on detecting the influences of the textile material 9 on the electrical field and to derive therefrom the physical properties of the textile material 9. The capacitive testing of textile material is sufficiently known from the state of the art. In the present embodiment, two electrodes 142.1, 142.3 are used as transmitter electrodes, and the other two electrodes 142.2, 142.4 as receiver electrodes.

Similar to the optical interface 55 and to the optical transmitter and receiver module 65, the device 1 according to FIG. 6 is provided with an electrical interface 155 and an electrical connecting part 165. The electrical interface 155 can be arranged as a plug-in connection, as adequately known from the field of electronics and as available on the market. Electrical components 164 are schematically shown in the electrical connecting part 165, which can be amplifiers, filters, modulators or demodulators for example. The electrical connecting part 165 is connected to the electronic unit 70 by means of one or several electrical conductors 171. Mechanical positioning means 53 can be provided for the optical transmitter and receiver module 65 and for the electrical connecting part 165, as described above.

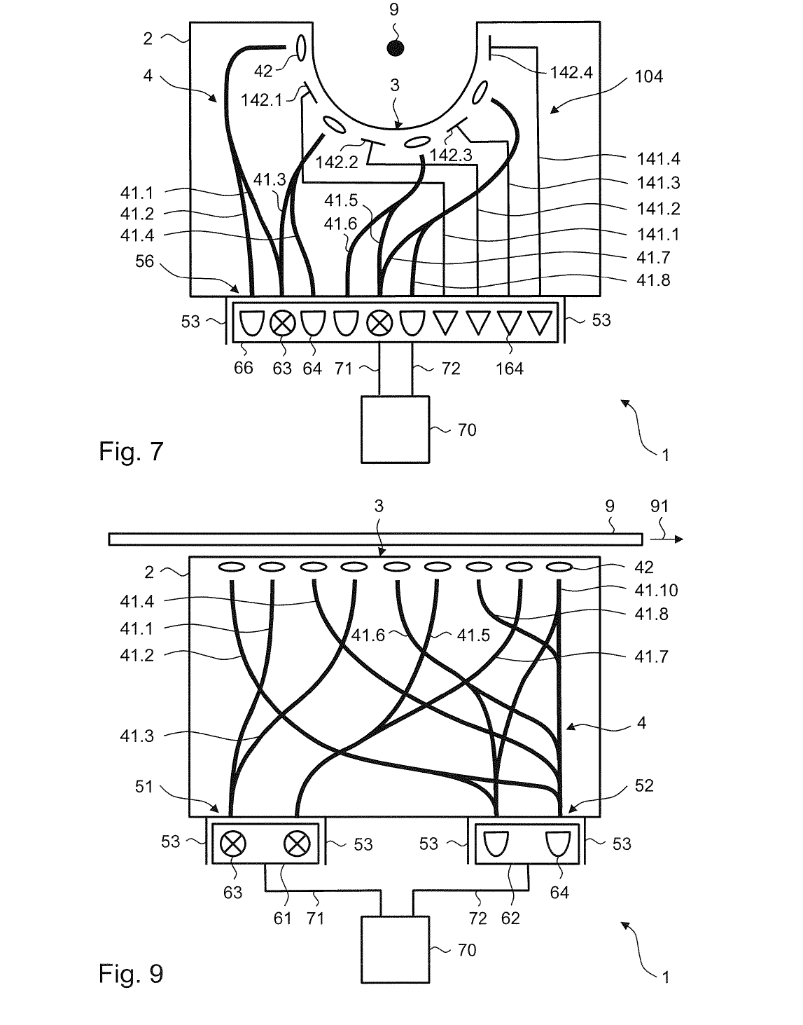

The sixth embodiment of the device 1 in accordance with the invention, which is shown in FIG. 7, substantially corresponds to that of FIG. 6. In the embodiment of FIG. 7, the substrate 2 comprises a combined optical-electrical interface 56 outside of the scanning region 3. Said optical-electrical interface 56 connects the optical waveguide structure 4 and the electrical conductor structure 104 to an optical-electrical connecting part 66. The optical-electrical interface 56 comprises mechanical positioning means 53 for positioning the optical-electrical connecting part 66 with respect to the substrate 2. The advantage of this embodiment over the one of FIG. 6 is that only one single interface 56 and only one single connecting part 66 are required. As already mentioned with respect to FIG. 6, two electrodes 142.1, 142.3 can be used as transmitter electrodes and the other two electrodes 142.2, 142.4 as receiver electrodes.

FIG. 8 shows a seventh embodiment of the device 1 in accordance with the invention. It concerns a combined integrated-optical and integrated-electrical device 1, as in FIG. 6. In contrast to the embodiment of FIG. 6, the light sources 63 and the light receivers 64 are not attached in this case to an external transmitter and receiver module but to the substrate 2. The light sources 63 and the light receivers 64 can be integrated as integrated-optical components on the substrate 2 or be applied as separate components to the substrate 2. Such components can alternatively be situated outside of the substrate 2, and the light can be incoupled into the illumination waveguides by means of respective coupling elements which are generally known or can be outcoupled from the detection waveguides.

The two light sources 63 and the two transmitter electrodes 142.1, 142.3 are triggered by a respective signal generator 167. The signal generators 167 are shown in FIG. 8 as microprocessors. The person skilled in the art of electronics knows many other types of signal generators. The microprocessors 167 are preferably integrated circuits in separate housings and are applied by means of a plug-in and/or solder connection to the substrate for example. Electronic components 167 such as amplifiers or filters can be provided between the signal generators 167 and the electrodes 142.1, 142.3, and possibly also the light sources 63. Preprocessing units 169 are respectively attached as close as possible behind the light receivers 64 or the receiver electrodes 142.2, 142.4, which are provided to preprocess the respective electrical output signals. Preprocessing can comprise preamplification, filtering and/or demodulation for example. The signals thus preprocessed are supplied to an electrical interface 155, which is preferably situated at an edge of the substrate 2. The interface 155 can simultaneously be used for supplying electrical signals such as control signals and electrical power to the electrical conductor structure 104. It can be arranged as a generally known electrical multiple plug.

FIG. 9 illustrates that the scanning region does not have to be semicircular, as shown in the previously discussed embodiments. In the eighth embodiment according to FIG. 9, a longitudinal axis and direction of movement 91 of the textile material 9 lies in the plane of the substrate 2 but outside of the substrate 2. The scanning region 3 coincides with a portion of a side of the rectangular substrate 2, and is arranged in a straight manner and parallel to the longitudinal axis 91 of the textile material 9. Focusing lenses 42 are arranged along the scanning region 3, preferably also situated on a straight line. They are situated at the orifices of waveguides 41.1 to 41.8 which form a waveguide structure 4. In the embodiment of FIG. 9, four illumination waveguides 41.1, 41.3, 41.5, 41.7 and five detection waveguides 41.2, 41.4, 41.6, 41.8, 41.10 are present. An emitter module 61 and a receiver module 62 are attached to another edge of the substrate 2. The right one of the two light receivers 64 attached to the receiver module 62 receives and adds light from all five detection waveguides 41.2, 41.4, 41.6, 41.8, 41.10, whereas the left light receiver 64 only receives and adds light from every other detection waveguide 41.2, 41.6, 41.10. Such an addition of light that was reflected at different, preferably equidistant, positions of the textile material 9 can supply additional information on the textile material 9. Apart from the arrangement of the scanning region 3, the position of the substrate 2 with respect to the longitudinal axis 91 of the textile material 9 and the waveguide structure 4, this embodiment is similar to that of FIG. 2, and the same reference numerals are used for elements that correspond to each other. Thus, further explanations are unnecessary here.

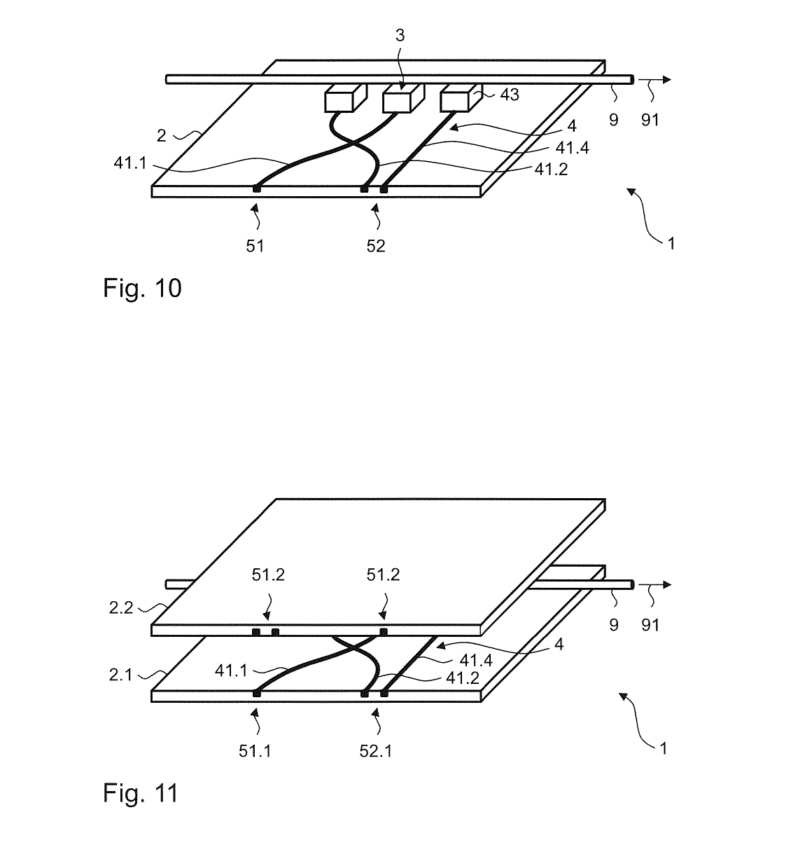

FIG. 10 shows a ninth embodiment of the apparatus 1 in accordance with the invention, in which the longitudinal axis 91 of the textile material 9 is situated parallel to the plane of the substrate 2, but is spaced therefrom. As a result, the textile material 9 is moved above the substrate 2 along its longitudinal direction 91. The scanning region 3 lies in or above the plane of the substrate 2. Light which is guided by means of an illumination waveguide 41.1 from an incoupling interface 51 to the scanning region 3 is outcoupled in the scanning region 3 towards the textile material 9. After interaction with the textile material 9, e.g. reflection and/or scattering on the same, at least a portion of said light is incoupled into the detection waveguides 41.2, 41.4 and guided therefrom to a outcoupling interface 52. Optical coupling elements 43 are required in this embodiment for incoupling and outcoupling the light in the scanning region 3, which coupling elements are capable of outcoupling light out of the plane of the substrate 2 or incoupling said light from the outside into a waveguide 41.2, 41.4 integrated on the substrate 2. Such coupling elements 43 are known and need not be discussed here in closer detail. The coupling elements 43 can additionally be equipped with focusing lenses and other optical components. The emitter module and the receiver module, the conductors and the electronic unit are not shown in FIG. 10 for reasons of simplicity of the illustration. They can be arranged in the same way as or similar to the preceding drawings.

In the embodiment of FIG. 11, the textile material 9 passes between two mutually spaced substrates 2.1, 2.2, wherein the substrate planes are situated in parallel with respect to each other and the longitudinal axis 91 of the textile material 9 is also situated in parallel with respect to the substrate planes. The substrates 2.1, 2.2 and the waveguide structures arranged thereon as well as the coupling elements 43 (not shown in FIG. 11) can be arranged similar to the substrate 2 in the embodiment according to FIG. 10. The advantage of the embodiment according to FIG. 11 is that light transmitted past the textile material 9 and/or through the textile material 9 can also be detected. For this purpose, an incoupling element on the second substrate 2.2 is assigned to an outcoupling element on the first substrate 2.1 in such a way that the incoupling element receives light of the outcoupling element and vice versa. Such transmission arrangements are used for determining a transverse dimensions and/or the hairiness of the textile material 9, whereas reflection arrangements according to FIG. 10 are used for the detection of foreign substances and/or optical properties of the textile material. Both substrates 2.1, 2.2 are equipped with incoupling interfaces 51.1, 51.2 and outcoupling interfaces 52.1, 52.2.

FIG. 12 shows an embodiment of the apparatus 1 in accordance with the invention with a flexible or bendable substrate 2. This property is utilized in this case in that the substrate 2 is not flat but bent into a cylinder jacket. In order to provide the device 1 with higher mechanical stability, it is advantageous to provide a support element 24 the substrate 2. In the embodiment of FIG. 12, the support element is arranged as a cylindrical tube 20, on the inner wall of which the bent substrate 2 is attached. The textile material 9 extends through the interior of the cylinder, preferably along a cylinder axis. The scanning region 3 extends along the circumference of the substrate 2 and can extend as required more or less in the axial direction. The textile material 9 can be scanned optically in this embodiment along its entire circumference. Several coupling elements 43, e.g. six thereof, are attached to the substrate 2 and are directed against the textile material 9, as already described with respect to FIGS. 10 and 11. At least one outcoupling element and at least one incoupling element are present on the coupling elements 43. Optical waveguides 41 are associated with the coupling elements 43, which waveguides can extend not only in the circumferential direction but also in the axial direction and are therefore only partly visible in the cross-sectional view of a FIG. 12.

It is understood that the present invention is not limited to the embodiments as discussed above. With knowledge of the invention, the person skilled in the art will be able to derive further variants which also belong to the subject matter of the present invention. In particular, the discussed embodiments can be combined with each other in an arbitrary fashion. Although many of the enclosed drawings are shown with axial-symmetric substrate forms and waveguide structures for aesthetic reasons, such symmetry is not necessary for the present invention. Asymmetric arrangements might be preferable in practice depending on the application. Furthermore, the number of light sources, light receivers, waveguides, ends of waveguides, lenses et cetera which are used in the drawings by way of example, shall in no way be understood as limiting.

### REFERENCE NUMBERS

1 Device
2 Substrate
20 Support element
3 Scanning region
31 Light beams
4 Optical waveguide structure
41 Optical waveguide
42 Focusing lens
43 Optical coupling element
51 Incoupling interface
52 Outcoupling interface
53 Mechanical positioning means
55 Optical interface
56 Optical-electrical interface
61 Emitter module
62 Receiver module
63 Light source
64 Light receiver
65 Transmitter and receiver module
66 Optical-electrical connecting part
70 Electronic unit
71, 72 Electrical lines
9 Textile material
91 Longitudinal axis and the direction of movement of the textile material
104 Electrical conductor structure
141 Electrical conductor path
142 Electrode
155 Electrical interface
164 Electrical component
165 Electrical connecting part
167 Signal generator

1. The use of a substrate with at least one optical waveguide structure integrated thereon for the optical inspection of a moving textile material.

2. The use according to claim 1, wherein an electrical conductor structure for the electrical inspection of the moving textile material is additionally integrated on the substrate.

3. A device for the optical inspection of a moving textile material, comprising:
a substrate on which a scanning region for optically scanning the textile material is provided, and
an optical waveguide structure that is integrated on the substrate and opens at least partly into the scanning region.

4. A device according to claim 3, wherein at least two optical waveguides of the optical waveguide structure open into the scanning region.

5. A device according to claim 4, wherein at least two waveguides are arranged for guiding light towards the scanning region, and at least two waveguides are arranged for guiding light away from the scanning region.

6. A device according to claim 5, wherein the orifices of the waveguides guiding towards and away from the scanning region are disposed adjacent to each other in an alternating fashion.

7. A device according to claim 3, wherein at least one orifice of a waveguide into the scanning region is provided with a focusing lens.

8. A device according to claim 3, wherein the optical waveguide structure comprises at least one junction with at least two branches.

9. A device according to claim 8, wherein at least two of the branches face the scanning region.

10. A device according to claim 8, wherein at least two of the branches face away from the scanning region.

11. A device according to claim 3, wherein the substrate outside of the scanning region comprises at least one optical interface for connecting the at least one optical waveguide structure to a respective optical connecting part.

12. A device according to claim 11, wherein an incoupling interface is provided for incoupling light into at least one optical waveguide structure and an outcoupling interface which differs from the incoupling interface is provided for outcoupling light from at least one optical waveguide structure.

13. A device according to claim 11, wherein the at least one optical interface comprises mechanical positioning means for positioning the optical connecting part with respect to the substrate.

14. A device according to claim 3, wherein an electrical conductor structure is additionally integrated on the substrate, which structure opens at least partly into the scanning region.

15. A device according to claim 14, wherein at least two electrical conductor paths of the electrical conductor structure open into the scanning region.

16. A device according to claim 15, wherein the orifices of the optical waveguides and the orifices of the electrical conductor paths are arranged adjacent to each other in an alternating fashion.

17. A device according to claim 14, wherein at least one orifice of an electrical conductor path is provided with an electrode in the scanning region.

18. A device according to claim 14, wherein the substrate comprises at least one electrical interface for connecting the at least one electrical conductor structure to a respective electrical connecting part outside of the scanning region.

19. A device according to claim 18, wherein the at least one electrical interface comprises mechanical positioning means for positioning the electrical connecting part.

20. A device according to claim 19, wherein the substrate comprises at least one optical-electrical interface for connecting the at least one optical waveguide structure and the at least one electrical conductor structure to an optical-electrical connecting part outside of the scanning region, and the at least one optical-electrical interface comprises mechanical positioning means for positioning the optical-electrical connecting part.
